(19) United States
(12) Patent Application Publication
(10) Pub. No.: US 2026/0137944 A1
(43) Pub. Date: May 21, 2026

(54) FLUOROLESS AND NON-CONTACT TEMPORARY PACING AND SHOCKING SYSTEM TO THE HEART

(71) Applicant: Mossaab Shuraih, Houston, TX (US)

(72) Inventor: Mossaab Shuraih, Houston, TX (US)

(21) Appl. No.: 19/446,608

(22) Filed: Jan. 12, 2026

Related U.S. Application Data
(63) Continuation of application No. PCT/US2024/037287, filed on Jul. 10, 2024.
(60) Provisional application No. 63/525,965, filed on Jul. 11, 2023.
(60) Provisional application No. 63/655,600, filed on Jun. 4, 2024.

(51) Int. Cl. A61N 1/362; A61N 1/02; A61N 1/04; A61N 1/05; A61N 1/372; A61N 1/39

(52) U.S. Cl. CPC A61N 1/3625; A61N 1/025; A61N 1/0408; A61N 1/0502; A61N 1/3621; A61N 1/37247; A61N 1/3904

(57) ABSTRACT

The hybrid stimulation system is an electrical stimulation system serves to delivered an electrical stimulation impulse to the heart (human or animal heart) , using a combination of internal (intravascular) and external (body surface) electrodes, the hybrid electrical stimulation system can be used for pacing of the heart during slow heart beat (bradycardia), or shocking the heart during fast heart beat (Tachycardia) , or to terminate fast abnormal heart beat (tachy-arrhythmia) by means of override pacing (Anti-tachycardia pacing). This hybrid system can be used and introduced into the body without the need for imaging guidance such as fluoroscopy or echocardiography to insert the internal component, and does not require direct contact between the internal pacing wire and the myocardium (internal heart surface).

### CROSS REFERENCE TO RELATED APPLICATIONS

This application claims the benefit of the filing date of U.S. provisional patent application Ser. No. 63/525,965, entitled “HYBRID ELECTRICAL STIMULATION SYSTEM TO THE HEART,” filed on Jul. 11, 2023; and U.S. provisional patent application Ser. No. 63/655,600, entitled “HYBRID ELECTRICAL STIMULATION SYSTEM TO THE HEART,” filed on Jun. 4, 2024; and is a continuation and claims the benefit of International PCT application serial number PCT/US2024/037287, entitled “HYBRID ELECTRICAL STIMULATION SYSTEM TO THE HEART,” filed on Jul. 10, 2024; the disclosures of which are incorporated herein by reference for all purposes.

### TECHNICAL FIELD

The technical field relates to the management of cardiac arrhythmia, slow heart beat “bradycardia” and/or fast heart beat “tachycardia”, specifically related to temporary pacing for bradycardiac arrhythmias, and/or shock therapy (defibrillation or electrical cardioversion) for tachycardiac arrhythmias.

### BACKGROUND INFORMATION

Arrhythmias are electrical disturbances in normal the heart electrical signal, these conditions can be life-threatening and can lead to what is known as “cardiac arrest” a situation where the heart effective pumping function is compromised resulting in limited blood flow or loss of blood flow to the body, including the brain which can cause loss of consciousness and even death—if not promptly treated.

Delivering electrical stimulation to the heart is a common medical practice and is used to manage patients with a very slow heartbeat (bradycardia), providing pacing therapy where a small electrical output is delivered to the heart to “capture” the heart muscle and increase the heart rate. Electrical stimulation is also used during a very fast heartbeat (Tachycardia) by delivering a high-energy shock to the heart to reset the tachycardia back into a normal rhythm.

The sinus node, also known as the sinoatrial (SA) node, is a small cluster of specialized cells located in the upper part of the right atrium of the heart. The SA node is often referred to as the natural pacemaker of the heart because it generates the electrical impulses that initiate each heartbeat. The signal from the SA node passes to the lower chambers of the heart (the ventricles) through another specialized tissue called the atrioventricular (AV) node.

Slow heartbeat, or bradycardia, can be caused by a variety of pathologies and diseases that affect either the SA node or the AV node, leading to sinus bradycardia or an AV block, respectively.

Treatment of the slow heartbeat, when it is not a reversible situation, may be performed by artificially delivering an electrical impulse or “pacing” to the heart through temporary or permanent pacemakers.

Ventricular arrhythmia is a type of abnormal heart rhythm that originates in the ventricles, the lower chambers of the heart. These arrhythmias are characterized by irregular electrical activity in the ventricles, which can lead to ineffective or chaotic contractions of the heart muscle. Ventricular tachycardia or ventricular fibrillation are types of ventricular arrhythmia that can be life-threatening tachyarrhythmias, and in an emergent situation, they are usually treated with a high-energy shock known as “Defibrillation.”

Another set of tachyarrhythmias that are regarded as more benign in nature such as atrial fibrillation, atrial flutter, and supraventricular tachycardias are not usually an immediate life-threatening situation, but they can be very serious and symptomatic and may require urgent interventions and, in many instances, they may also be managed with some form of electrical shock via a process known as “Cardioversion.”

Cardiac stimulation, which includes pacing, defibrillation or cardioversion, are presently performed through internal, i.e., intravascular devices (temporary or permanent pacing or defibrillator leads), or external devices through the use of skin surface patches (or in some special situations, defibrillation through implantable extravascular permanent defibrillator with external lead under the skin may also be used).

Delivering electrical stimulation to the heart is a common medical practice used to manage patients with bradycardia (slow heartbeat) and tachycardia (fast heartbeat). For bradycardia, pacing therapy involves delivering a small electrical output to the heart to stimulate the heart muscle and to increase the heart rate.

For tachycardia, electrical stimulation can be used to deliver a high-energy shock to reset the heart back to a normal rhythm. In some instances, tachycardia can be treated by fast pacing of the heart for a brief period of time to terminate the arrhythmia. This procedure, known as anti-tachycardia pacing therapy, is incorporated into conventional implantable cardiac defibrillators.

Bradycardia is a slow heartbeat most commonly caused by sinus node dysfunction or atrioventricular block, and in an emergent situation requires the use of a temporary pacemaker system to be inserted to deliver small electrical stimulation to the heart to keep the heart beating at an acceptable normal rate, until the problem is resolved or a permanent pacemaker is inserted.

External pacemakers are frequently used by medical personnel because external pacing pads are easy to apply and can be used quickly. However, they can be unreliable in many situations, depending on the patient's anatomy, the clinical scenario, or different patient's body habitus, the external pacing stimulation may not be sufficient to evoke electrical capture in the heart muscle in what known as “fail to capture” the heart, and in emergent situation temporary pacemaker could be lifesaving measure, and failure to capture can result in bad clinical outcomes. In addition, due to the nature of the external pacing, a high energy output is required to capture the heart externally, and that type of pacing is usually associated with significant discomfort from the patient feeling the electrical stimulation and the chest muscle's forceful contractions.

A second type of temporary pacemaker system, which may be more reliable to capture the heart, uses an internal temporary pacer wire (as used in this disclosure, the term “wire” is not limited to a single conductor and may include an assembly of separate wires or conductors e.g., a lead). The temporary wire or lead is usually inserted under fluoroscopic guidance into the heart chambers and normally placed in the right ventricle. Pacing with internal wires is generally more reliable to capture the heart tissue as the lead is usually in direct contact with the internal surface of the heart muscle. Therefore, smaller energy output is needed which would rarely cause a discomfort sensation (especially in comparison to the external high energy output pacing). Furthermore, temporary lead may be more reliable than the external patches in achieving the capturing of the heart tissue, they are more likely to successfully pace the heart and less likely to exhibit fail-to-capture since the wire is placed inside the heart chamber.

In most cases the insertion of a temporary wire is somewhat time-consuming process in situation where fast rapid pacing to the heart is needed to save the patient's life because the placement of temporary wires is usually performed under fluoroscopy in a radiology suit or in a Cath-lab setting. Cath lab activation can be a time-consuming and costly process, and when it is needed outside of regular working hours it necessitates calling-in extremely skilled medical physicians, staff nurses and technicians. Thus, a Cath lab procedure can significantly prolong the time where the patient is not being effectively paced. The longer the patient goes without successful pacing, the higher the risk for serious complications and potential bad outcome damage to vital organs such as the brain or kidneys.

In some emergent cases situations when a Cath lab is not readily available, the medical personal may attempt to place a temporary wire blindly (without fluoroscopy) into the right ventricle (RV). Unfortunately, a blind placement usually requires multiple attempts to place the lead successfully in the right ventricle and it can be unsuccessful process and, in some cases can cause complications like perforation of the heart wall.

Yet, another problem with the temporary passive fixation pacing wires is that they are prone to movement and dislodgment and that can lead to loss of capture—which can happen when patients move or during patient's transportation or spontaneously. A dislodged temporary wire may need repositioning that may again require fluoroscopy and Cath lab utilization.

What needed is a pacing and shocking system that can be effective and reliable in pacing and or shocking the heart, the system needs to be easy and fast to apply where medical staff with minimal training should be able to use and install quickly and safely. The desired system should be inserted and applied to the patient with or without the need for imaging such as fluoroscopic or echocardiographic visualization, saving valuable time and resources.

### SUMMARY

In response to these and other problems, in one embodiment, there is a hybrid electrical stimulation system which may be an electrical circuit comprised of internal and external electrode components, delivering electrical impulse between the surface and the internal components will direct the vector of the electrical stimulation towards the heart mass, rending the system more efficient than just external pacemaker or shocking system.

The invention provides electrical stimulation of the heart where the electrical circuit has both internal and external electrical components. the external component can be in one embodiment detachable surface electrodes such as patches or pads, and the internal components in one embodiment can be a wire carrying number of electrodes, such as a wire with distal tip electrode and more proximal coil electrode located at short distance away from the tip. The internal component is inserted through a percutaneous trans vascular access. The internal wire does not necessary require direct contact with the myocardium for the appropriate function of the system.

These and other features, and advantages, will be more clearly understood from the following detailed description taken in conjunction with the accompanying drawings. It is important to note the drawings are not intended to represent the only aspect of the invention.

### BRIEF DESCRIPTION OF THE DRAWINGS

FIG. 1 is a conceptual diagram illustrating one embodiment of a hybrid electrical stimulation system.

FIGS. 2A and 2B are conceptual illustrations of stimulation vectors which may be produced by the embodiment of FIG. 1.

FIGS. 3A and 3B are conceptual illustrations of sensing vectors which may be produced by the embodiment of FIG. 1.

FIG. 4 is a functional diagram of one embodiment of a control panel and/or pulse generator which may be used in the embodiment of FIG. 1.

FIG. 5 is a simplified schematic illustration of one embodiment of sensing circuit which may be incorporated into the control panel and/or pulse generator of FIG. 4.

FIGS. 6A, 6B, and 6C are conceptual illustrations of various configurations possible with one embodiment of an output vector switch circuit which may be used in the control panel and/or pulse generator of FIG. 4.

FIG. 7 is a conceptual illustration of one embodiment of an input switch circuit for the sensing circuit of FIG. 5.

FIG. 8 is a conceptual illustration of a proximal end internal wire design of one embodiment that may be used with the embodiment of FIG. 1.

FIGS. 9A to 9D are conceptual illustrations of various alternative embodiments of a proximal end wire showing the relative placement of the embodiments relative to a human heart of a patient.

### DETAILED DESCRIPTION

For the purposes of promoting an understanding of the principles of the present inventions, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the inventions as described herein are contemplated as would normally occur to one skilled in the art to which the invention relates.

Specific examples of components, signals, messages, protocols, and arrangements are described below to simplify the present disclosure. These are, of course, merely examples and are not intended to limit the invention from that described in the claims. Well-known elements are presented without detailed description in order not to obscure the present invention in unnecessary detail. For the most part, details unnecessary to obtain a complete understanding of the present invention have been omitted inasmuch as such details are within the skills of persons of ordinary skill in the relevant art. Details regarding control circuitry or mechanisms used to control the function of the various elements described herein are omitted, as such control circuits are within the skills of persons of ordinary skill in the relevant art.

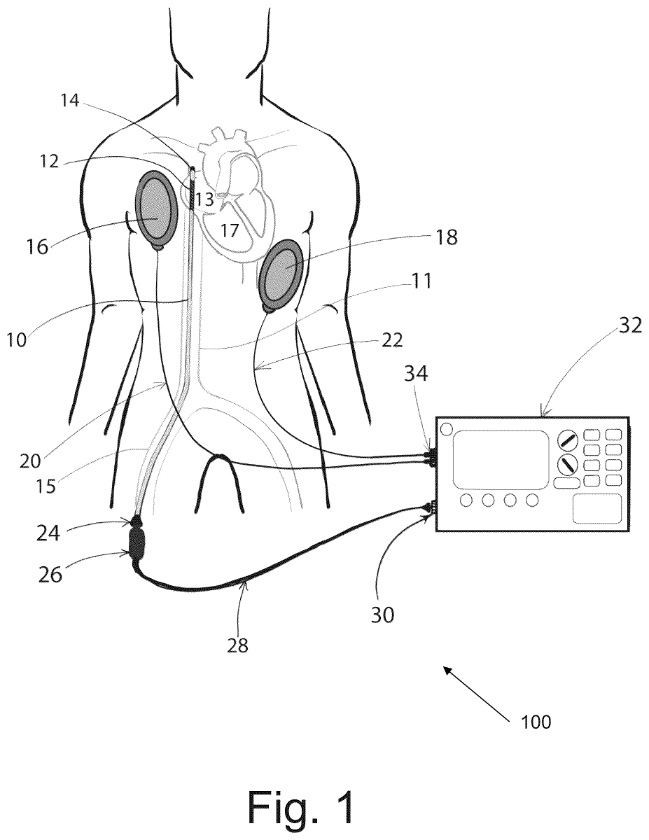

FIG. 1 represents one embodiment of the hybrid stimulation system 100 that is comprised of two external skin pads or external patches 16 and 18, and internal (intravascular) lead or wire 10. The internal component (in this embodiment, the internal wire 10) may have a distal tip electrode 14 located at a distal tip of the wire 10 and a more proximal coil electrode 12 positioned relatively close to the distal tip (i.e., an electrode array assembly). The delivery of the electrical impulse, for pacing or shocking, would take place between the external electrodes (skin patches 16, 18) and the internal electrodes (the internal wire 10).

The delivery of an electrical impulse, for pacing or shocking, may take place between the external patches 16 and 18, and the internal wire 10. In certain embodiments, the internal wire 10 will direct a vector of the electrical simulation towards the patient's heart or heart mass. Because the internal wire is closer to the heart than external patches, the system 100 may be more effective at capturing the heart than traditional external patches, where the electrical circuit forms between the two external patches.

In certain embodiments, the internal electrodes 12 and 14 and the external electrodes on the pads 16 and 18 are connected to a control panel/pulse generator 32 which, in certain embodiments, functions as a control unit of the system. The external patches 16 and 18 or external electrode assembly may be placed on the patient's chest wall in this embodiment in an anterior and posterior position, where each patch is connected with a separate connecting cable 20, 22 to the control panel/pulse generator 32 through input/output ports 34 for the external electrodes. In certain embodiments, the internal wire 10 is inserted through a femoral vein access 15, and it passes through the inferior vena cava 11 to the right atrium 13 of the patient's heart as illustrated in FIG. 1.

In certain embodiments, the internal wire 10 has a distal tip electrode 14, and proximal coil electrode 12, the internal wire 10 may be inserted under sterile conditions then connected to an extension cable 28, via a connecting electrical plug within a connecting port 24 positioned at a proximal end of the internal wire 10. In certain embodiments, the connecting port 24 couples to a cable connecting port 26 positioned at the distal end of the extension cable 28. The extension cable 28 may then connect to the pulse generator 32 through an input/output port 30 for the internal electrodes.

In certain embodiments of this system 100, any two or more sets of electrodes may be configured to form a pacing or shocking circuit. As discussed above, pacing circuits typically have lower energy output and usually the output may be continuous at a set frequency. In contrast, a shocking or defibrillating circuit generates a relatively high energy electrical output meant to be delivered at single time (which can be repeated as necessary).

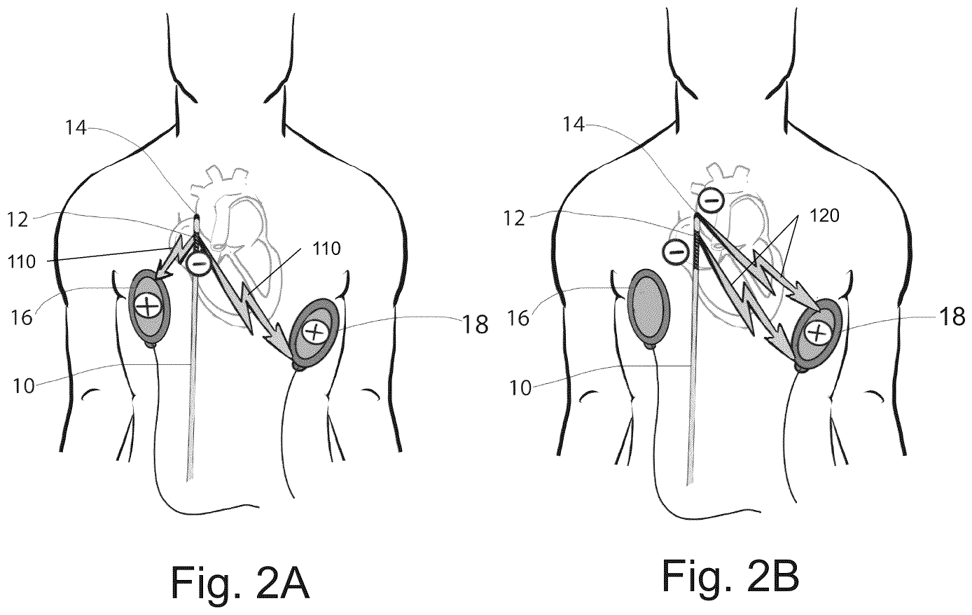

FIGS. 2A and 2B illustrate two examples of stimulation vectors that can be delivered through certain embodiments of the hybrid stimulation system 100. In the illustrated example of FIGS. 2A and 2B there are two external patches 16 and 18, and the internal wire 10 coupled two electrodes: the distal tip electrode 14 and proximal coil electrode 12. FIG. 2A illustrates a stimulation vector 110 where the proximal coil electrode 12 functions as the negative pole (the cathode) of the circuit, and the positive end (the anode) is comprised of the two external patches 16 and 18 working in combination.

In contrast, FIG. 2B illustrates a different stimulation arrangement where the combination of the distal tip electrode 14 and the proximal coil electrode 12 function as the cathode and one of the surface patches 18 functions as the anode to produce a stimulation vector 120.

In certain embodiments, different vector activation configurations can be used and can be selected by the user through a control panel interface which allows the user to determine the most effective vector to delivered the desired therapy whether pacing or defibrillation. In certain embodiments, different vectors configuration and different polarities can be selected between any two or more electrodes in the system, different combinations of vectors such as internal electrode to one patch or vice versa or between the internal electrodes or between the external patches, switched cathode and anode configuration. As discussed above, a shocking/defibrillation output would be at higher magnitude than the pacing output. The shocking output would be a single shock that can be repeated as required while pacing output may be delivered continuously at fixed interval.

In certain embodiments, the hybrid system 100 may be able to “sense” the internal cardiac signals using different sensing vectors that utilize different electrodes combinations as discussed below. For instance, when advancing the wire 10 from a peripheral venous access like using the femoral vein 15 (FIG. 1) through the inferior vena cava 11 to the right atrium 13, the system 100 can sense internal cardiac signals once the internal electrodes 12 and 14 are in close proximity to the heart.

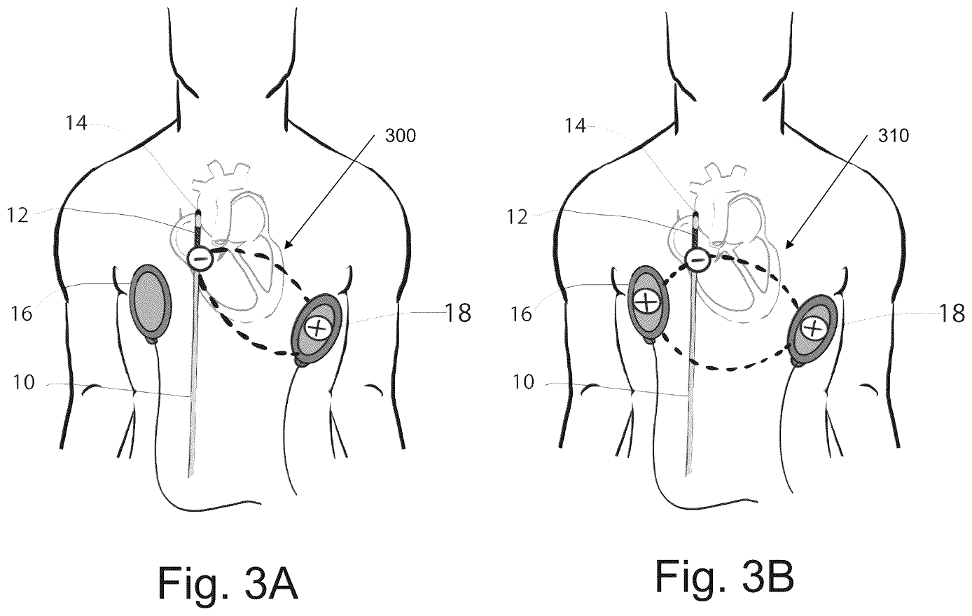

FIGS. 3A and 3B illustrate exemplary embodiments of sensing circuits formed when using with the hybrid electrical system 100. In certain embodiments, the sensing circuit would utilize two or more electrodes of the system. Hybrid sensing with internal and external combination may create a sensitive circuit with a relatively larger “antenna” to pick up the internal cardiac signals. Note that although such a large sensing area is more prone to pick up noise signals, the large sensing area also enables the system 100 to recognize proximity to the heart. Additionally, internal sensing of the cardiac signal between the internal electrodes 12 and 14 can be also be performed and that sensing may be more sensitive to a cardiac near field signal.

In certain embodiments, sensing the cardiac signal allows appropriate functioning of the whole stimulation system, and it can also be used as an indicator of the appropriate placement of the internal wire 10 when advancing the wire in a straight manner through the transcutaneous vascular access.

FIG. 3A illustrates one example of sensing circuit 300 between the proximal coil electrode 12, positioned in the right atrium, and one of the external patches (e.g., the external patch 18). In contrast, FIG. 3B illustrates an alternative sensing circuit 310 formed between the proximal coil electrode 12 at one end (representing the negative pole) and the two external patches 16 and 18, combined to form the positive pole of the circuit.

In certain embodiments, different circuit configurations and/or different circuit polarities encompassing any two or more electrode combinations in the system 100 can be selected using the user interface. These configurations can also be tweaked to achieve an optimal sensing configuration or circuit.

In certain embodiments, the internal wire 10 may have a blunt flexible atraumatic tip that can be advanced in a straight manner to the heart to the right atrium 13 without the need for imaging guidance such as fluoroscopy or cardiac ultrasound until the distal tip of the wire reaches close to the heart and ideally inside the right atrium 13. Sensing the cardiac signal allows for a determination of the appropriate functioning of the whole stimulation system. Furthermore, it can also be used as an indicator of appropriate placement of the internal wire, as the wire can be advanced until sensing the intrinsic cardiac signal is achieved, with the combination of ability to capture the heart with pacing stimulation that will indicate satisfactory positioning of the internal wire 10, the position of which can be adjusted by slight advancing or pulling the internal wire 10. The internal wire 10 does not need to be in direct contact with the myocardium, as positioning in close proximity to heart chambers, as in this embodiment, which is inside the right atrium, enables the system 100 to sense and deliver pacing between the external and the internal electrodes.

In certain embodiments, the hybrid system 100 may be controlled by the control panel/pulse generator 32, which enables the user to select the type of therapy needed (i.e., pacing, or shocking and the parameters related to the selected therapy). In certain embodiments, the control panel/pulse generator 32 may be any hardware/software combination that is able to generate electrical output and be able to sense the heart intrinsic signal, and thus, modify the sensing and the output parameters.

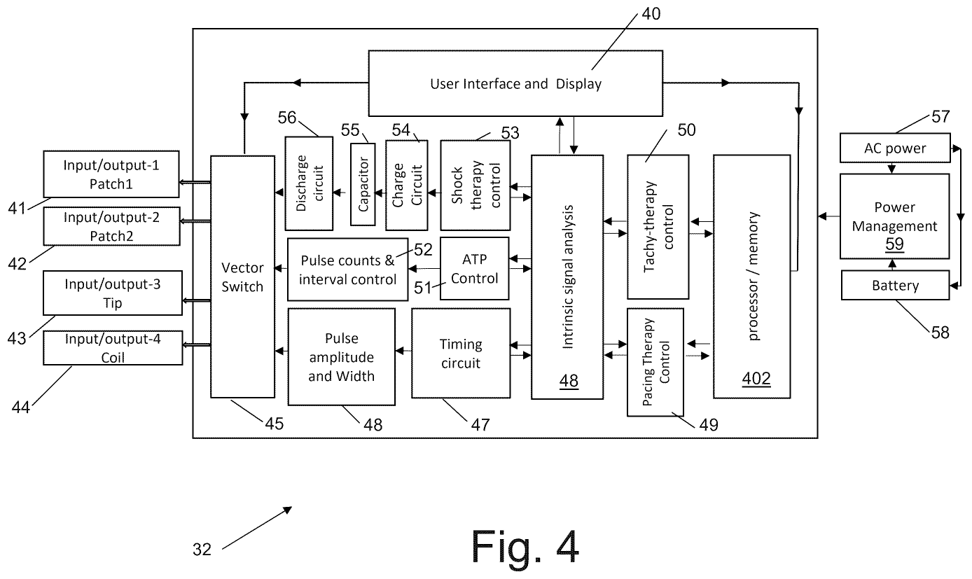

FIG. 4 illustrates one embodiment of a simplified schematic representation of some of the main and basic circuits for the output circuits in the pulse generator/control panel 32. In other embodiments, these circuits may be represented by software processes or modules and/or one or more processors performing these functions. A processor, for instance, processor 402, may contain or be in communication with a medium, such as computer memory, for storing instructions. In certain embodiments, the processor 402 may be one or more microprocessors, application-specific integrated circuits (ASICs), field programmable logic arrays (FPGAs), digital signal processors (DSPs), or other equivalent integrated or discrete logic circuitry (whether in firmware, software, or hardware).

In the illustrative embodiment of FIG. 4, a power source, such as an AC power outlet 57 or a rechargeable battery 58, may be coupled to a power management circuit 59, which manages and supplies the electrical power to the control panel/pulse generator 32.

A user interface and information display 40 is provided to allow a user to interact with the system and receive information about the heart and the device function. Any electronic analogue or digital system interface that allows a user to control the system may be used and is included in the scope of this disclosure.

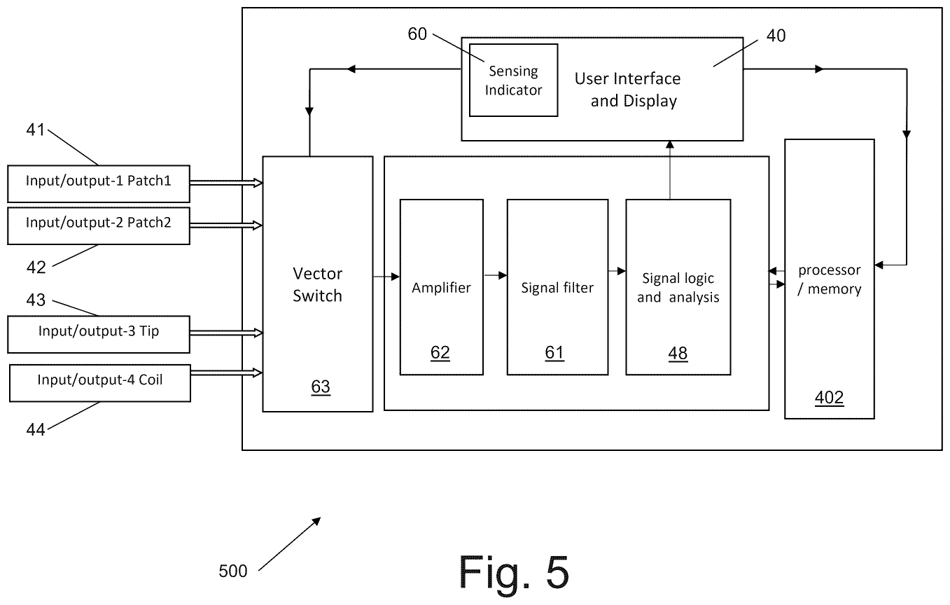

In certain embodiments, the user interface 40 may have a display panel or screen that receives and displays the input from an intrinsic signal analysis unit 48. The intrinsic signal analysis unit 48 may be connected or in electrical communication with a sensing circuit (explained in FIG. 5 below), and may provide information to the therapy circuits about the internal cardiac electrical activity of the patient (such as the heart rate, regularity, and other intrinsic information). In certain embodiments, the information received can be displayed to the user on the user interface 40. The information displayed may be, for example, the patient's rhythm, heart rate, and/or the patient's electrocardiogram (ECG or EKG).

Through the user interface 40, the user can select a specific therapy (i.e., pacing or shocking). In a situation where the patient is experiencing tachycardiac arrhythmia (such as ventricular tachycardia or atrial fibrillation) and the patient requires tachy-arrhythmia treatment, the user can activate a tachy therapy control circuit 50. The tachy therapy control circuit 50 may use information from the intrinsic signal analysis circuit 48 to direct the tachy-therapy based on the user's selection (e.g., the user can set the system to either deliver an electrical shock or deliver anti-tachycardia pacing). A shock therapy control unit 53 can then send an input signal to a charger circuit 54 that will initiate charging a capacitor 55 to a specific chosen output value selected by the user via the user interface 40 (for example, charge to deliver a 200 Joules shock). Once a discharge command is activated by the user through the user interface 40, the capacitor 55 will then discharge through a discharge circuit 56 into a vector switch circuit 45 that will channel the output into output channels, which are connected to the selected electrodes of the hybrid system 100. For instance, there may be four output channels in total: an input/output channel 41 for external patch 16, an input/output channel 42 for external patch 18; an input/output channel 43 for the distal tip electrode 14, and an input/output channel 44 for the proximal coil electrode 14. Other embodiments may have more or less electrodes and the circuit will have corresponding number of input/output channels. The electric stimulation can have different configuration using any combination of two or more input/output channels that can be assigned as anode (positive) and the cathode (negative) parts of the overall circuit as explained above.

A vector switch circuit 45 may be controlled by the user via the interface 40 to predefine which electrodes of the system 100 will be used as cathode or anode, using a dial or selectable menu options on the user interface 40. In certain embodiments, the user can preselect, for example, a delivery vector: output channel 41 (external patch 16) to output channel 44 (proximal coil 12). If that delivery vector fails to convert the patient, the user can adjust the shock vector to utilize the combined output channels 41 and 42 (for external patches 16 and 18) as an anode, and the channel 44 as a cathode, which was discussed in reference to FIG. 2A. In certain embodiments, the polarity and the vector of the shock or the pacing output can also be adjusted. Additional details on the vector switch circuit 45 will be discussed below.

In certain embodiments, if the charged capacitor 55 receives a command to abort delivery of the shock, it will discharge into the aborting circuit (not shown in FIG. 4).

In situations where the user chooses to treat a stable tachycardia with override pacing anti-tachycardia pacing therapy (ATP), the user may engage an ATP control circuit 51. In certain embodiments, the ATP control circuit 51 may determine, based on the intrinsic heart rate, the interval between the pacing output to be delivered, the pulse counts. An interval circuit 52 may then deliver a set number of fast pacing output, again channeled through the output vector switch circuit 45. For example, if the patient has stable ventricular tachycardia at heart rate of 172 beats per minute, the user can choose to pace the heart at a slightly faster rate (for example, at a heart rate of 190 beats per minute) for a predetermined number of beats (e.g., 5-12) at a time to terminate the tachycardia.

In certain embodiments, a pacing therapy control circuit 49 may be activated from the user interface 40 to deliver pacing therapy for brady arrhythmia such as sinus bradycardia or AV nodal block. In such situations, the user may select a desired heart rate beats per minute (bpm). A timing circuit 47 may then generate an impulse at a predetermined interval (e.g., every 60000/Set Heart Rate millisecond). For example, if the user selects a desired heart rate to be 60 beats per minute, a timer in a timing circuit 47 will deliver an output signal every (60000/60) or 1000 milliseconds. The timer can be reset if an intrinsic signal is sensed from the intrinsic signal analysis unit 48 in a situation where the pacing mode is set to synchronize with the intrinsic patient heart rate. Otherwise, with asynchronous pacing mode, the timer may deliver an output signal at fixed set intervals regardless of the intrinsic cardiac electrical activities. A pacing mode (e.g., synchronized or asynchronized) can be selected from the user interface 40. Once selected, a mode signal may be delivered into a pacing control circuit 49.

Output from the pacing control circuit 49 may be characterized by an amplitude (voltage) and width (duration) that may be determined by a pulse amplitude and width circuit 46. The pulse amplitude and width circuit 46 may deliver an output signal to the vector switch circuit 45, which in turn can control the preselected output channels to form a pacing circuit. For example, pacing the heart may be achieved by forming a circuit between the proximal coil electrode 12 and the two external patches 16 and 18 as discussed in reference to FIG. 2A. In other words, the output channel 44 may be selected so that the proximal coil electrode 12 will function as a cathode and both output channels 41 and 42 may be selected so that the external patches 16 and 18 combine to function as an anode.

FIG. 5 is a conceptual functional diagram of an exemplary sensing circuit 500, which may be incorporated into the Control panel/Pulse generator 32. As illustrated, signals from the various electrodes (e.g., external patches 16,18, distal tip electrode 14, and proximal coil electrode 14) may use output channels 41,42,43,44 described above as input channels for sensing the intrinsic cardiac signals. In alternative embodiments, different numbers of channels may be based on different numbers and configurations of electrodes.

The configuration of the sensing circuit 500 may be selected based on user preferences using the user interface 40, which will send command signals to a sensing vector switch circuit 63, which then configures the appropriate choice of positive and negative poles of the circuit. The input signals from the electrodes (the sensing signals) may be amplified via an amplifier unit 62 and then processed with a signal filter unit 61 that will pass the filtered signal to an intrinsic signal analysis unit 48. The intrinsic signal analysis unit may provide data and sensing signals to the therapy circuits described above in reference to FIG. 4. In certain embodiments, in addition to displaying the data from the intrinsic signals on the user interface 40, other information may also be displayed. For example, the rhythm ECG could also be displayed.

In certain embodiments, the user interface 40 may also have a sensing indicator 60, which determines whether there is a satisfactory positioning of the internal wire when the internal wire is used to sense the intrinsic heart signals. For example, in situations where the sensing circuit uses the proximal coil electrode 12 as a cathode (therefore, the sensing input channel will be an input/output channel 44) and the combination of the two external patches 16 and 18 to be the positive pole (anode), the sensing input channel for these external patches would be channels 41,42. As discussed above with respect to FIG. 3B, this configuration of a larger circuit of sensing with a large antenna field enables the user to detect the intrinsic heart signal once the internal wire 10 is in a desired position and making the placement of the internal wire 10 possible without a need for imaging guidance such as fluoroscopy, X-ray or Echocardiographic guidance. Additional confirmation of an appropriate positioning of the wire 10 may be the ability to start to capture the heart tissue using such a configuration.

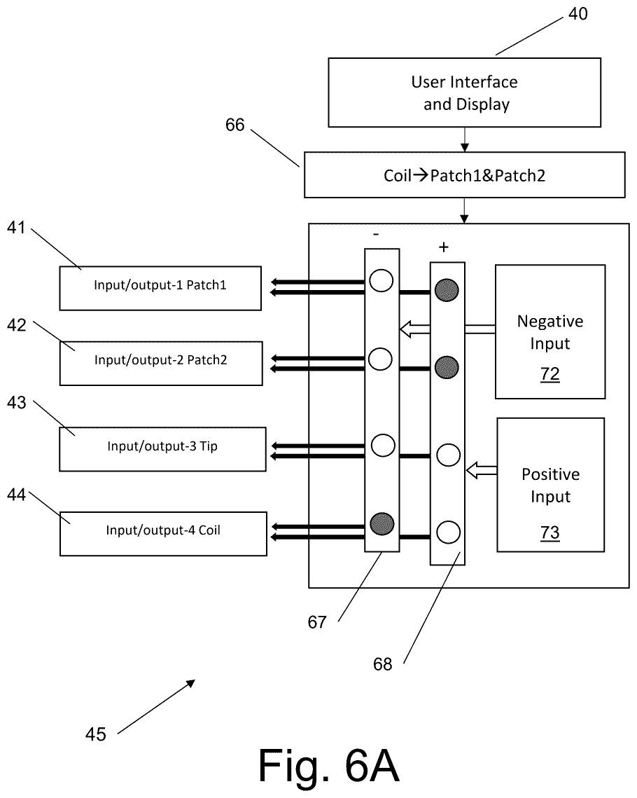

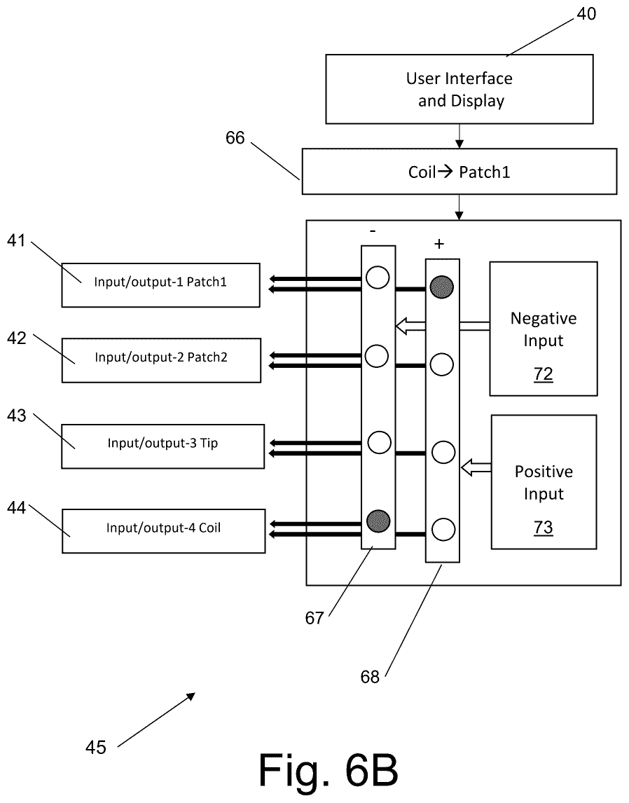

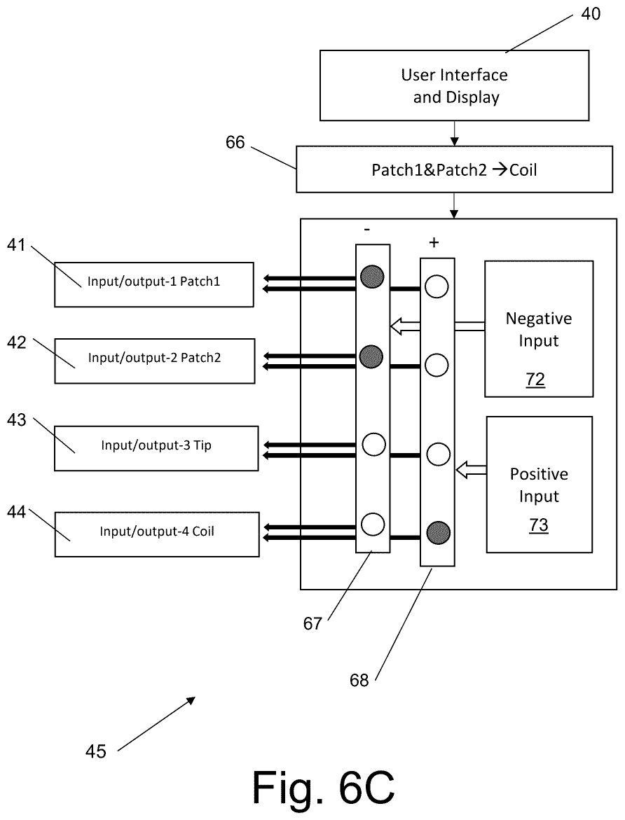

FIGS. 6A through 6C are functional diagrams that illustrate the basic functionality of the output vector switch circuit 45 (see FIG. 4) in one exemplary embodiment, which may be incorporated into the control panel/pulse generator 32. The output vector switch circuit 45 controls which output channels will be assigned as cathode (negatively charged electrode) vs anode (positively charged electrode). This configuration is achieved by connecting the output channel of each electrode into two separate electric switch circuits: a negative switch circuit 67, and a positive switch circuit 68. In certain embodiments, the negative switch circuit 67 and the positive switch circuit 68 are able to render active or inactive status into each output channel connected to respective switch circuits 67 and 68.

Such functionality may be achieved by different means known in the art, such as mechanical or digital. In this illustrative embodiment, switch circuits are used that function as demultiplexer circuits. Demultiplexer (DEMUX) electrical circuits are used in electronic devices to route a specific selected input to one or more of several outputs.

In certain embodiments, the user interface 40 may have a switch dial or selection menu for selecting the output configuration, which in the examples illustrated by FIGS. 6A through 6B, such functionality is represented by the vector selection switch 66. FIG. 6A illustrates an embodiment where there are four electrodes in use: two surface patches 16, 18; the internal distal tip electrode 14, and the proximal coil electrode 12, which are connected to four output channels, 41,42,43, and 44, respectively.

In this configuration of the system and in the example shown in FIG. 2A, the cathode was selected to be the proximal coil electrode 12, and the anode was selected to be the combination of the two surface patches 16 and 18. FIG. 6A illustrates the circuit arrangement for this specific configuration, where each of the four output channels depicted in this configuration is connected to two separate output demultiplexer switch circuits: a negative switch circuit 67 and a positive switch circuit 68.

In this illustrative embodiment, a negative impulse output 70 and the positive impulse output receive electrical energy from the power management circuit 59. The negative impulse output 70 in turn feeds the negative switch circuit 67, while the positive impulse output 71 feeds the positive switch circuit 68. For instance, when the user selects the proximal coil electrode 12 and the two external patches 16, 18 configuration to deliver the output (as shown in FIG. 2A) the negative switch circuit 67 will assign the coil output channel 44 as active channel (depicted in gray circle versus inactive channel depicted as a white circle inside the negative switch circuit 67), and the positive switch circuit 68 will assign both output channels 41 and 42 to be active. Thus, this exemplary configuration will channel the positive output of the circuit 45 from the positive output source 71 to the output channel of patches 41 and 42, while channeling the negative charges from the negative output source 70 to the coil electrode output channel 44.

FIG. 6B is a functional diagram that demonstrates different configuration of the vector switch circuit 45 where the impulse output is delivered between the coil channel 44 which is assigned as cathode, by being active in the negative switch circuit 67 while the channel 41 for the external patch 16 is assigned as anode because it is active in the positive switch circuit 68 as illustrated in FIG. 6B.

FIG. 6C is a functional diagram that demonstrates another configuration similar to the configuration of FIG. 6B, but where the polarities are reversed. In FIG. 6C, the output channel 44 for the proximal coil electrode 12 is assigned as anode (illustrated as active in the positive switch circuit 68), while the combination of the output channels 41 and 42 for the external patches 16 and 18 is assigned as a cathode (illustrated as active in the negative switch circuit 67).

Thus, as illustrated in FIGS. 6A through 6C, the vector switch circuit 45 allows for different combinations of electrode selection and different polarity selections (e.g., reversing anode and cathode assignments) between any combination of the electrodes that are used by the system 100.

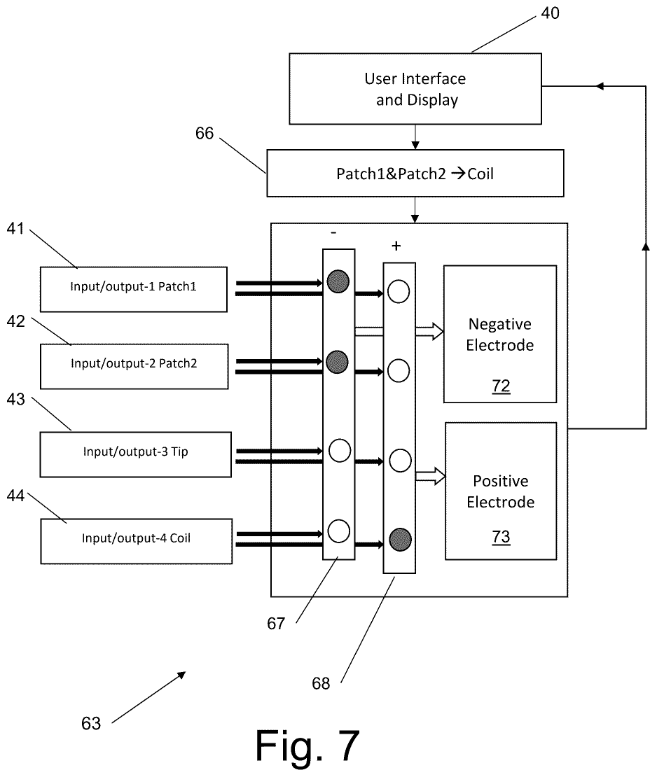

FIG. 7 is a functional diagram that illustrates a vector sensing circuit 63 that can be configured to utilize different combinations of electrodes in the system 100. In certain embodiments, the physical circuit 63 may be the same or similar to the vector switch circuit 45 described above. In certain embodiments, the input channels 41, 42, 43, and 44 are the same as the output channels in the circuit 45. However, in this illustrative circuit, they function to receive the input from the electrodes used in the system 100, using a multiplexer electrical switch to channel different inputs into one output, the final input from the sensed signal will be transmitted to a positive electrode 73, or a negative electrode 72, parts of the sensing circuit. The output from the positive electrode 72 and or the negative electrode 73 feeds the sensing circuit illustrated in FIG. 5 where the sensed signal go through amplifier 62 and filter 61.

In this embodiment, the system 100 includes two external patches and two internal electrodes: coil and tip, 41,42,43, and 44 are the input channels (same as channels used to deliver output), the user can select a sensing configuration using the sensing-vector selection switch 66 (or a menu on the interface).

In FIG. 7, for example, the user assigned a sensing circuit configuration where the negative pole of the sensing circuit receives a feed or the intrinsic heart electrical activation signal from the electrodes of skin patches 16 and 18 via channels 41 and 42, and the positive input receives input from the coil channel 44. (In pacemakers and defibrillators, the devices are designed to sense the heart's own electricity or intrinsic signal. This signal is also used to generate an electrocardiogram.)

In this example, the input channels 41 and 42 are illustrated as the circles in the FIGS. 6A and 7 where the dark circles indicate that the circuit is active, (i.e., the dark circles in 67 in FIG. 7 is active) the negative switch circuit 67 are rendered active, while the rest of the input channels are rendered inactive, the negative input signal then is passed through to the negative electrode channel of the negative output source 70 (FIGS. 6A-6C). This information is then processed by the sensing circuit and may be displayed as a heart EKG or heart rate on the user interface.

At the same time, the positive switch circuit 68 will activate the coil input channel 44 and will inactivate the rest of the input channels. The positive input signal is then passed to the positive electrode channel of the positive output source 71 (FIGS. 6A-6B).

The sensing circuit configuration and polarity can be changed by the user interface 40 with a selection menu or dial 66 that can assign different part of the circuit to sense the intrinsic cardiac signal in specific polarity to determine the configuration to best sense cardiac signal because the heart signal is very small and it may be sensed best in one configuration vs another depending on the individual patient characteristics and the clinical situation.

The internal component or wire 10 and the associated electrodes can be placed through percutaneous vascular access, the wire 10 can be advanced with or without fluoroscopic guidance to a position adjacent to the heart (ideally into the right atrium “RA”), as the wire 10 is just required to be advanced in a straight manner. Therefore, the wire 10 can be advanced without using fluoroscopy, rendering fluoroscopy or other imaging optional. In such embodiments, the internal wire distal tip may be a flexible blunt tip as known in the art.

The correct positioning of the wire in the heart chambers or in a close proximity to the heart can be confirmed with sensing the intrinsic cardiac electric signal as discussed above. Sensing the intrinsic cardiac signal will indicate that the internal electrodes are in close proximity to the heart and in a good position. The vector sensing circuit 63 discussed above can be configured to complete the circuit using signals between the distal tip electrode 14 and the proximal coil electrode of the internal wire 10. In yet other embodiments, the vector sensing circuit 63 may be configured to complete the circuit using signals running between the skin patches 16 and 18 and the internal electrodes 12 and 14.

Another indicator of appropriate positioning of the wire is when achieving capture during pacing of the heart, with pacing vector between the external patches and the internal electrodes (for example patch-to-coil, patch-to-tip, patch-to-tip/coil), once the wire is in a close proximity to the heart the pacing vector will incorporate cardiac mass and start to capture the heart tissue.

FIG. 1 demonstrates one embodiment of this system, where quick femoral vein access can be obtained and the internal wire can be advanced in a straight manner without fluoroscopy to the systemic circulation. To approach the heart chamber, in this embodiment, the wire is placed in the right atrium (RA).

If the internal wire moves for any reason or another, adjustment of the internal wires can be done easily and safely at the bedside by slightly sliding the wire in or out of the position in a straight manner. The wire can also be manipulated under fluoroscopy to place it into a specific cardiac chamber, for example, the wire can be curved manually and inserted under fluoroscopy to reach specific heart chambers such as the right ventricle or the coronary sinus if clinically required.

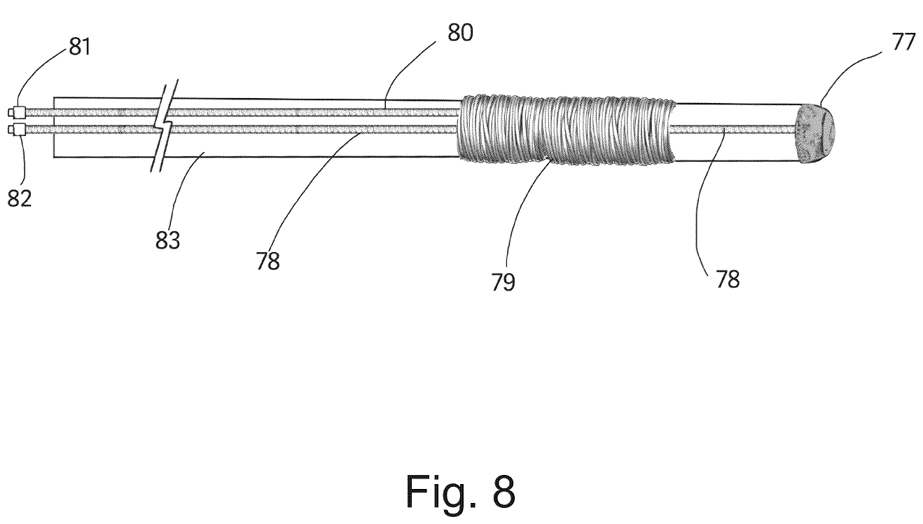

FIG. 8 is an illustration of one embodiment showing the basic components of the internal wire 10, the distal tip electrode 14, and the proximal coil electrode 12. In certain embodiments, the distal tip electrode 14 and the proximal coil electrode 12 can be made from a conductive material, such as platinum group metal alloys, such as a platinum-iridium alloy or other conductor material, such as sterling silver or any other appropriate conductive material.

In certain embodiments, the distal tip electrode 14 is connected to a conductor cable 78, and the proximal coil electrode 12 is connected to a conductor cable 80. In certain embodiments, the conductor cables 78 and 80 can be made from platinum materials. In certain embodiments, the conductor cables 78 and 80 may be surrounded and insulated with an insulation material 83 that separates the two conductor cables 78 and 80 and also insulates the system from the environment. In certain embodiments, the insulation material 83 can be formed of silicone or polyurethane materials or any other appropriate insulation material with flexibility and durability. Each of these conductor cables 78 and 80 has a connection point 81 and 82, respectively, which are located at the proximal end of the cables. The conductor cables 78 and 80 may be connected to the output/input port 30 (FIG. 1) of the control panel/pulse generator 32 - either through a direct connection to the pulse generator, or through an extension cable 28 as illustrated in FIG. 1.

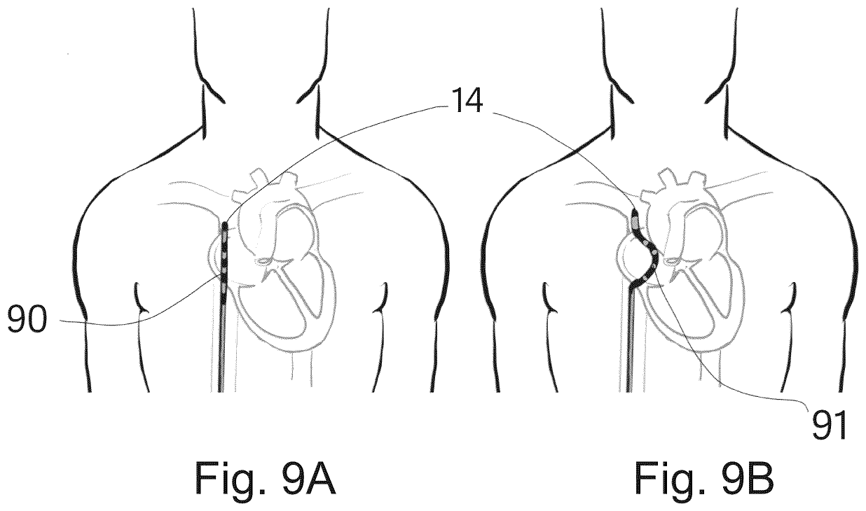

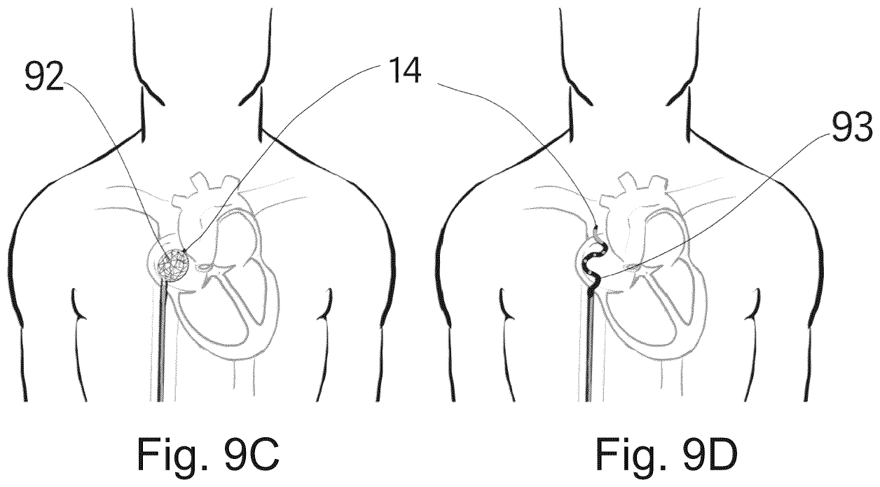

FIGS. 9A-9D are illustrations showing alternative embodiments of the internal wire 10 with various electrode configurations. FIG. 9A illustrates the internal wire 10 with multiple electrodes 90 rather than the proximal coil 12. FIG. 9B illustrates a curved end of the internal wire 91, which could include a single coil or multiple separated electrodes. FIG. 9C illustrates a spherical proximal electrode 92 that can be formed with spherical expandable material. FIG. 9D illustrates an internal wire with multiple curves or a multiple curved end 93.

### ALTERNATIVES AND DIFFERENT EMBODIMENTS

As previously discussed, FIG. 1 illustrates one possible embodiment of the hybrid stimulation system 100, where the system includes external patches 16 and 18, and the internal wire 10 with the distal tip electrode 14 and proximal coil electrode 12. The internal wire 10 and its associated electrodes 14 and 12 is illustrated inserted through the femoral vein 15 into the right atrium 13. However, FIG. 1 illustrates only one insertion configuration. The internal wire 10 may be inserted into veins or arteries. Transcutaneous vascular access can be any central or peripheral vascular access venous or arterial, other vein such as subclavian or jugular veins can be used, peripheral arterial access such as femoral arty as well where the internal wire can be situated in the descending aorta for example. As discussed above, FIG. 1 is illustrated one embodiment of the wire positioned in the right atrium, through femoral vein access, but other embodiments for placing the wire can be any peripheral vascular access, venous or arterial also is contained in this invention, the wire or the internal component can be placed in any vascular structure close to the heart, or inside any heart chamber, placing the wire straight with venous access will give the advantage of positioning without necessary needing fluoroscopic guidance, even with arterial access if that was to be desired/or needed, placing the wire in straight manner can land it in the aorta behind the heart which could be acceptable position, but further advancement of the wire in the arterial system may require fluoroscopic visualization to avoid potential complications.

The internal component represented in FIG. 1 is a straight wire with two distal electrodes tip and proximal coil other embodiments of the internal component would be more than one wire with different electrodes arrangement where hybrid pacing or shocking can utilize any electrode combinations.

The shape of the internal electrodes includes other possible shapes rather than just the tip and the coil, other different embodiments could include, but not limited to a wire with multiple electrodes as illustrated in FIG. 9A, where the wire has multiple electrodes constellation in the proximal position of the multiple electrodes 90. FIG. 9B has a curved end of the internal wire with coil or multiple electrodes in the curved end of internal wire 91, the curved end can be a single curve or multiple curves as illustrated in FIG. 9D with the multiple curved end 93 that can be spiral curves on either a 2D or 3D plane. FIG. 9C shows an alternative embodiment with spherical expandable electrode 92 made with expandable conductor mesh that can be expanded and collapsed.

The different shapes of the internal component may have different favorable pacing or shocking results, as they provide a larger surface to channel the electrical stimulation towards the heart mass, and they can provide contact with the myocardium, which is helpful but not a mandatory requirement for the appropriate functioning of the hybrid stimulation system. The internal wire 10 design is not meant to be limited to a single conductor. The internal wire 10, for instance, can have a central lumen to enable placement over a guide wire. In yet other embodiments, there may be an inflatable balloon at the tip of the wire 10 for easier atraumatic introduction of the wire into the vascular space.

The external component in FIG. 1 is represented by detachable two pads or patches 16 and 18. However, other embodiments may include other forms of surface electrodes and different numbers of electrodes and surface patches.

The control panel or the external pulse generator 32 may include any hardware/software combination able to sense and analyze intrinsic signals and able to generate different electrical outputs in different configurations of polarity between the internal and external electrodes, such as a computer, laptop, mobile device, handheld tablet, or dedicated pulse generator.

### ADVANTAGES

In summary, one advantage of certain aspects is the ability to stimulate the heart between skin surface electrodes and internal electrodes, providing the effectiveness of the internal pacing/shocking therapy with the advantage of the quick external pacing/hocking therapy, in a hybrid manner. Furthermore, using an internal component that can be inserted in a straight manner provides the option to place the internal wire without fluoroscopic or imaging guidance, which is usually required for the current internal pacing or shocking devices. Moreover, the internal wire does not need to be in direct contact with the internal heart surface, which is a requirement for current internal pacing devices that are usually wedged against the ventricular or atrial cavity. The hybrid system may also require less energy output compared to the pure external pacing system, as the internal component will channel the electrical impulse directly towards the heart mass, making it more tolerated by patients.

The abstract of the disclosure is provided for the sole reason of complying with the rules requiring an abstract, which will allow a searcher to quickly ascertain the subject matter of the technical disclosure of any patent issued from this disclosure. It is submitted with the understanding that it will not be used to interpret or limit the scope or meaning of the claims.

Any advantages and benefits described may not apply to all embodiments of the invention. For the United States national phase, when the word “means” is recited in a claim element, Applicant intends for the claim element to fall under 35 USC 112(f). Often a label of one or more words precedes the word “means”. The word or words preceding the word “means” is a label intended to ease referencing of claims elements and is not intended to convey a structural limitation. Such means-plus-function claims are intended to cover not only the structures described herein for performing the function and their structural equivalents, but also equivalent structures. For example, although a nail and a screw have different structures, they are equivalent structures since they both perform the function of fastening. Claims that do not use the word “means” are not intended to fall under 35 USC 112(f).

The foregoing description of the embodiments of the invention has been presented for the purposes of illustration and description. It is not intended to be exhaustive or to limit the invention to the precise form disclosed. Many combinations, modifications and variations are possible in light of the above teaching. For instance, in certain embodiments, each of the above-described components and features may be individually or sequentially combined with other components or features and still be within the scope of the present invention. Undescribed embodiments which have interchanged components are still within the scope of the present invention. It is intended that the scope of the invention be limited not by this detailed description, but rather by the claims.

For instance, in some embodiments, there may be an ability to stimulate the heart with hybrid circuit between skin surface electrodes component and internal electrodes component placed inside the vascular system or inside one of the heart chambers.

In other embodiments, there may be a hybrid temporary pacemaker using external body surface electrodes and internal electrodes components (internal refers to inside one of the heart chambers or within the intravascular space).

In certain embodiments, a temporary pacemaker wire can be inserted in a straight manner into the intravascular space without need for fluoroscopic guidance, confirmation of the position with sensing intrinsic cardiac signal and/or ability to pace between the temporary wire and surface electrode.

In certain embodiments, there may be a hybrid shocking device to deliver electrical shock to the heart for cardioversion or defibrillation between skin surface electrodes such as a patch and internal electrodes component such as a coil, the internal component can be placed inside one of the heart chambers or vessels or within the intravascular space close to the heart.

In certain embodiments, a hybrid shocking system can be inserted in a straight manner into the intravascular space without need for fluoroscopic guidance, confirmation of the position with sensing intrinsic cardiac signal and/or pacing between the internal wire and surface electrodes.

In certain embodiments, an external pulse generator may be able to trigger electrical stimulation between skin surface electrode and internal electrodes, and able to sense intrinsic cardiac signal between the internal and/or external electrodes joining the second individual conductor to the second ring electrode.

In yet other embodiments, there may be medical electrical stimulation system, comprising: at least one external electrode assembly adapted to be attachable to a surface of a patient; an internal electrode array assembly positioned proximal to a distal end of a wire wherein the wire is adapted to be percutaneously inserted into a patient; and a controller in electrical communication with the at least one external electrode and the internal electrode array.

The embodiments can further comprise an additional external electrode assembly adapted to be attachable to a surface of a patient.

In some embodiments, the internal electrode array comprises at least two electrodes where one electrode is positioned at a distal tip of the wire and a second electrode is positioned longitudinally along the wire.

In some embodiments, the second internal electrode is in a form of a coil.

In some embodiments, the controller comprises: at least one processor; a pacing module in communication with the at least one processor, wherein the pacing module is adapted to deliver electrical stimulation to the patient at a predetermined amplitudes and a predetermined frequency; a shock module in communication with the at least one processor, wherein the shock module is adapted to deliver a single electrical stimulation to the patient; and a user interface in communication with the at least one processor, wherein the user interface includes a selector for allowing a user to select between delivering electrical stimulation through either the pacing module or the shock therapy module.

In certain embodiments, the controller comprises: a tachy-therapy control unit which is communicatively coupled to a shock therapy control unit to deliver a single pulse at a predetermined amplitude; a pacing therapy control unit in communication with a timing circuit to deliver a pulse at a predetermined magnitude and width and the pacing therapy control unit is in communication with an anti-tachycardia pacing therapy control unit for determining pulse counts and interval control; a vector switch which receives signals generated as a result of the tachy-therapy control unit and the pacing therapy control unit to determine a pulse output delivered to a variety of output channels; a sensing circuit for determining internal cardiac activity; and a user interface for displaying internal cardiac activity and allowing a user to the control tachy-therapy control unit and the pacing therapy control unit.

In some embodiments, the sensing circuit comprises: a vector switch for receiving cardiac activity signals from electrode assemblies; an amplifier coupled to the vector switch for amplifying the received signals; a filter in communication with the amplifier for removing extraneous noise from the received signals; and an analysis module for interpreting the received signals and sending display signals to the user interface.

In certain embodiments, there may be a method of forming a defibrillating circuit, the method comprising: placing an external electrode assembly adjacent to a patient's skin; percutaneously introducing an internal electrode assembly into a patient's vein or artery and placing an internal electrode assembly within a proximity to a patient's heart; and directing an electrical pulse to produce a stimulation vector between the internal electrode assembly and the external electrode assembly.

In certain embodiments, there may be a method of forming a pacing circuit, the method comprising: placing an external electrode assembly adjacent to a patient's skin; percutaneously introducing an internal electrode assembly into a patient's vein or artery and placing an internal electrode assembly within a proximity to patient's heart; and directing a plurality of electrical pulses to produce stimulation vectors between the internal electrode assembly and the external electrode assembly.

In certain embodiments, there may be a method of determining an adequate placement of an internal electrode assembly comprising: placing an external electrode assembly adjacent to a patient's skin; percutaneously introducing an internal electrode assembly into a patient's vein or artery; advancing the internal electrode assembly towards the patient's heart until intrinsic cardiac signals can be obtained; and sensing internal cardiac signals by establishing a circuity between the external electrode assembly and the internal electrode assembly to determine if the internal electrode assembly is adequately placed in proximity to the heart to produce a stimulation vector to the patient's heart.

In some of the above methods, the placing the external electrode assembly comprises placing a first external electrode at a first position on a patient's skin and placing a second external electrode at a second position on a patient's skin.

In some of the above methods, the placing the internal electrode assembly comprises placing a first internal electrode at a tip of an electrical wire and placing a second internal electrode at a proximal distance from the first electrode along the wire.

Some of the above methods include selecting at least one electrode as a cathode and at least one electrode as an anode.

Some of the above methods include selecting the second internal electrode as a cathode and selecting the first external electrode and the second external electrode in combination to form an anode.

Some of the above methods include selecting the first internal electrode and the second internal electrode in combination to form a cathode and selecting the first external electrode as an anode.

In certain embodiments, there may be a system comprising: a means for placing an external electrode assembly adjacent to a patient's skin; a means for percutaneously introducing an internal electrode assembly into a patient's vein or artery and placing an internal electrode assembly within a proximity to a patient's heart; and a means for directing an electrical pulse to produce a stimulation vector between the internal electrode assembly and the external electrode assembly.

In certain embodiments, there may be a system comprising: a means for placing an external electrode assembly adjacent to a patient's skin; a means for percutaneously introducing an internal electrode assembly into a patient's vein or artery and a means for placing an internal electrode assembly within a proximity to patient's heart; and a means for directing a plurality of electrical pulses to produce stimulation vectors between the internal electrode assembly and the external electrode assembly.

In certain embodiments, there may be a system for determining an adequate placement of an internal electrode assembly comprising: a means for placing an external electrode assembly adjacent to a patient's skin; a means for percutaneously introducing an internal electrode assembly into a patient's vein or artery; advancing the internal electrode assembly towards the patient's heart until intrinsic cardiac signals can be obtained; and a means for sensing internal cardiac signals by establishing a circuity between the external electrode assembly and the internal electrode assembly to determine if the internal electrode assembly is adequately placed in proximity to the heart to produce a stimulation vector to the patient's heart.

In some of the above systems, the means for placing the external electrode assembly comprises a means for placing a first external electrode at a first position on a patient's skin and a means for placing a second external electrode at a second position on a patient's skin.

In some of the above systems, the means for placing the internal electrode assembly comprises a means for placing a first internal electrode at a tip of an electrical wire and a means for placing a second internal electrode at a proximal distance from the first electrode along the wire.

Some of the above systems include a means for selecting at least one electrode as a cathode and at least one electrode as an anode.

Some of the above systems include a means for selecting the second internal electrode as a cathode and a means for selecting the first external electrode and the second external electrode in combination to form an anode.

Some of the above systems include a means for selecting the first internal electrode and the second internal electrode in combination to form a cathode and a means for selecting the first external electrode as an anode.

What is claimed is:

1. A medical electrical stimulation system, comprising:
at least one external electrode assembly adapted to be attachable to a surface of a patient;
an internal electrode assembly positioned proximal to a distal end of a wire wherein the wire is adapted to be percutaneously inserted into a patient; and
a controller in electrical communication with the at least one external electrode and the internal electrode assembly,
the controller, including a sensing circuit, having logic to:
(1) detect intrinsic cardiac signals by forming a sensing vector between the internal electrode assembly and the external electrode assembly, and
(2) determine whether the internal electrode assembly is adequately positioned in proximity to a patient's heart to enable therapeutic stimulation.

2. The system of claim 1, wherein the logic to determine if the internal electrode assembly is adequately positioned includes logic to determine that the internal electrode assembly is adequately placed when the sensed intrinsic cardiac signals exceed a predetermined characteristic or threshold indicative of proximity to the patient's heart sufficient to permit therapeutic stimulation.

3. The system of claim 1, wherein the logic to determine if the internal electrode assembly is adequately positioned includes logic to determine whether heart tissue of the patient is able to be captured.

4. The system of claim 1, further comprising an additional external electrode assembly adapted to be attachable to a surface of a patient.

5. The system of claim 4, wherein the internal electrode assembly comprises one electrode positioned at a distal tip of the wire.

6. The system of claim 4, wherein the internal electrode assembly comprises one electrode positioned longitudinally along the wire at a proximal distance from a distal tip of the wire.

7. The system of claim 4, wherein the internal electrode assembly comprises at least two electrodes where one internal electrode is positioned at a distal tip of the wire and a second internal electrode is positioned longitudinally along the wire.

8. The system of claim 7, wherein the second internal electrode is in a form of a coil.

9. The system of claim 4, wherein the controller comprises:
at least one processor;
a pacing module in communication with the at least one processor, wherein the pacing module is adapted to deliver electrical stimulation to the patient at a predetermined amplitudes and a predetermined frequency;
a shock module in communication with the at least one processor, wherein the shock module is adapted to deliver a single electrical stimulation to the patient; and
a user interface in communication with the at least one processor, wherein the user interface includes a selector for allowing a user to select between delivering electrical stimulation through either the pacing module or the shock module.

10. The system of claim 4, wherein the controller comprises:
a tachy-therapy control unit which is communicatively coupled to a shock therapy control unit to deliver a single pulse at a predetermined amplitude;
a pacing therapy control unit in communication with a timing circuit to deliver a pulse at a predetermined magnitude and width and the pacing therapy control unit is in communication with an anti-tachycardia pacing therapy control unit for determining pulse counts and interval control;
a vector switch which receives signals generated as a result of the tachy-therapy control unit and the pacing therapy control unit to determine a pulse output delivered to a variety of output channels;
a sensing circuit for determining internal cardiac activity; and
a user interface for displaying internal cardiac activity and allowing a user to the control tachy-therapy control unit and the pacing therapy control unit.

11. The system of claim 1, wherein the sensing circuit comprises:
a vector switch for receiving cardiac activity signals from electrode assemblies;
an amplifier coupled to the vector switch for amplifying the received signals;
a filter in communication with the amplifier for removing extraneous noise from the received signals; and
an analysis module for interpreting the received signals and sending display signals to a user interface.

12. A method of determining an adequate placement of an internal electrode assembly comprising:
placing an external electrode assembly adjacent to a patient's skin;
percutaneously introducing an internal electrode assembly into the patient's vein or artery;
advancing the internal electrode assembly towards a heart of the patient until intrinsic cardiac signals can be obtained;
forming a sensing vector between the internal electrode assembly and the external electrode assembly;
detecting intrinsic cardiac electrical signals using a sensing circuit comprising an amplifier, a filter, and an analysis module; and
determining that the internal electrode assembly is adequately placed when the sensed intrinsic cardiac signals exceed a predetermined characteristic or threshold indicative of proximity to the patient's heart sufficient to permit therapeutic stimulation.

13. The method of claim 12, wherein the placing the external electrode assembly comprises placing a first external electrode at a first position on a patient's skin and placing a second external electrode at a second position on a patient's skin.

14. The method of claim 13, wherein the placing the internal electrode assembly comprises placing a first internal electrode at a tip of an electrical wire and placing a second internal electrode at a proximal distance from the first electrode along the wire.

15. The method of claim 14, further comprising selecting at least one electrode as a cathode and at least one electrode as an anode.

16. The method of claim 14, further comprising selecting the second internal electrode as a cathode and selecting the first external electrode and the second external electrode in combination to form an anode.

17. The method of claim 14, further comprising selecting the first internal electrode and the second internal electrode in combination to form a cathode and selecting the first external electrode as an anode.

18. A method of forming a defibrillating circuit, the method comprising:
placing an external electrode assembly adjacent to a patient's skin;
percutaneously introducing an internal electrode assembly into a patient's vein or artery;
placing an internal electrode assembly within a proximity to a patient's heart;
establishing a stimulation vector between the internal electrode assembly and the external electrode assembly; and
delivering a defibrillation pulse through the stimulation vector.

19. A method of forming a pacing circuit, the method comprising:
placing an external electrode assembly adjacent to a patient's skin;
percutaneously introducing an internal electrode assembly into a patient's vein or artery;
placing an internal electrode assembly within a proximity to patient's heart;
establishing a pacing vector between the internal electrode assembly and the external electrode assembly; and
delivering a plurality of pacing pulses through the pacing vector.
